(19) United States
(12) Patent Application Publication
(10) Pub. No.: US 2008/0194787 A1
(43) Pub. Date: Aug. 14, 2008

(54) Low-Viscosity Uretdion Group-Containing Polyaddition Compounds, Method Of Production And Use Thereof

(76) Inventors: Volker Weiss, Haltern am See (DE); Thomas Weihrauch, Dulmen (DE); Werner Grenda, Herne (DE); Silvia Herda, Herne (DE); Klaus Behrendt, Herne (DE)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/909,549

(22) PCT Filed: Jan. 27, 2006
(86) PCT No.: PCT/EP2006/050487

§ 371 Date: Sep. 24, 2007

(30) Foreign Application Priority Data
Mar. 23, 2005 (DE) 10 2005 013 401.7

(51) Int. Cl. C08G 18/70; C07C 263/00

(52) U.S. Cl. 528/67; 564/106

(57) ABSTRACT

The invention relates to low-viscosity polyaddition compounds containing uretdione groups, preparation process, and use.

The invention relates to low-viscosity polyaddition compounds containing uretdione groups, preparation process, and use.

Polyaddition compounds containing uretdione groups are known.

DE 101 470 describes reaction products of aromatic diisocyanates containing uretdione groups, and difunctional hydroxyl compounds.

DE 952 940, DE 968 566, and DE 11 53 900 describe reaction products of diisocyanates, diisocyanates containing uretdione groups, and difunctional hydroxyl compounds.

DE 20 44 838 claims the onward reaction of polyurethane compositions containing uretdione groups with polyamines.

DE 22 21 170 describes the reaction of NCO-terminated polyurethane compositions containing uretdione groups with diamines with preservation of the uretdione groups.

DE 24 20 475 contains the description of a process for preparing powder coating crosslinkers which are composed of diisocyanates containing uretdione groups, diisocyanates, and difunctional hydroxyl compounds.

U.S. Pat. No. 4,496,684 mentions reaction products of diisocyanates containing uretdione groups, and difunctional hydroxyl compounds, which are then intended for subsequent crosslinking with acid anhydrides.

A process for preparing polyaddition compounds containing uretdione groups is described in EP 269 943.

EP 601 793 describes one-part adhesives comprising polyisocyanates containing uretdione groups, polyisocyanates, and polyols.

EP 640 634 describes polyaddition compounds containing uretdione groups and further containing isocyanurate groups.

EP 1 063 251 describes a process for preparing polyaddition compounds containing uretdione groups. In that process, diisocyanates and polyisocyanates containing uretdione groups are mixed.

A feature common to all of these preparation processes and products is that during the solvent-free preparation at relatively high temperatures (>50° C.) the use of customary catalysts, dibutyltin dilaurate (DBTL) for example, for accelerating the reaction leads to unwanted side reactions (allophanates). The allophanates formed raise the melt viscosity of the resultant polyaddition compounds containing uretdione groups, and at the same time valuable reactive uretdione is destroyed. The raising of the melt viscosity is detrimental to the processing properties of such systems, in their utility as powder coating hardeners, for example. Powder coating hardeners of high viscosity are less easy to mix with other powder coating constituents, and on the coating surface lead to defects owing to inadequate flow.

It was an object of this invention to find polyaddition compounds containing uretdione groups that have significantly lower melt viscosities, and also a process for preparing them.

Surprisingly it has been found that the additional use of 0.1-5% by weight of carboxylic acids leads to significantly lower melt viscosities in the solvent-free preparation of polyaddition compounds containing uretdione groups at temperatures above 50° C. Significantly reduced means that the melt viscosity of the resultant product has fallen by at least 40% in comparison to the conventionally employed dibutyltin dilaurate under otherwise identical conditions (reaction temperatures and reaction times). The melt viscosity is dependent on the glass transition temperature. There are products available on the market with a low Tg (40-50° C.) (viscosity 30-300 Pas at 120° C.) and also products with a high Tg (70-80° C.) (viscosity 3000-20000 Pas at 120° C.). The baseline viscosity of these products is already drastically different. In comparison with the conventional mode of preparation, nevertheless, a significant decrease in melt viscosity can be expected in each case.

The invention provides low-viscosity polyaddition compounds containing uretdione groups and obtained by solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
   in which R=alkyl radical having 1 to 10 carbon atoms and X=carboxylate radical of a carboxylic acid having 1 to 20 carbon atoms and n=1, 2 or 3, m=1, 2 or 3 and n+m=4,
   in a concentration of from 0.01 to 3%, based on the total composition weight %; and
D) in the presence of monocarboxylic, dicarboxylic or polycarboxylic acids in a concentration of 0.1%-5% by weight, based on polyol B);
E) and/or, optionally, further aromatic, aliphatic, (cyclo-)aliphatic and/or cycloaliphatic polyisocyanates;
F) and optionally further monoalcohols, monoamines, diamines and/or blocking agents;
   wherein further auxiliaries and additives may be present.

The low-viscosity polyaddition compounds of the invention, containing uretdione groups, generally possess viscosities that are 40% lower than in the case of conventional products, normally in the range from 30 Pas (Tg 40° C.) to 20000 Pas (Tg 80° C.), measured in each case at 120° C.

Suitable starting materials for the polyisocyanates A) containing uretdione groups are aromatic, aliphatic, (cyclo-)aliphatic and/or cycloaliphatic polyisocyanates having at least two NCO groups, particularly the following: isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate/2,4,4-trimethylhexamethylene diisocyanate (TMDI), norbornane diisocyanate (NBDI), toluidine diisocyanate (TDI), and/or methylenediphenyl diisocyanate (MDI), and also tetramethylxylylene diisocyanate (TMXDI) are used with preference. Very particular preference is given to IPDI, HDI, and H₁₂MDI.

Polyisocyanates containing uretdione groups are well known and are described for example in U.S. Pat. No. 4,476,054, U.S. Pat. No. 4,912,210, U.S. Pat. No. 4,929,724, and EP 417 603. A comprehensive review of industrially relevant processes for dimerizing isocyanates to uretdiones is provided by J. Prakt. Chem. 336 (1994) 185-200. The reaction of isocyanates to uretdiones generally takes place in the presence of soluble dimerization catalysts, such as dialkylaminopyridines, trialkylphosphines, phosphoric triamides, triazole derivatives or imidazoles. The reaction—carried out optionally in solvents but preferably in the absence of solvents—is arrested by addition of catalyst poisons on attainment of a desired conversion. Excess monomeric isocyanate is separated off subsequently by means of short-path evaporation. If the catalyst is sufficiently volatile the reaction mixture can be freed from catalyst in the course of monomer separation. In that case there is no need to add catalyst poisons.

The dimerization of H₁₂MDI has been described only recently in WO 04005363 and WO 04005364.

Suitable compounds B) include all polyols (polyols are all compounds having at least two alcohol groups) commonly used in PU chemistry, with a molecular weight of at least 32.

The monomeric diols are, for example, ethylene glycol, triethylene glycol, butane-1,4-diol, pentane-1,5-diol, hexane-1,6-diol, 3-methylpentane-1,5-diol, neopentyl glycol, 2,2,4(2,4,4)-trimethylhexanediol, and neopentyl glycol hydroxypivalate.

The monomeric triols are, for example, trimethylolpropane, ditrimethylolpropane, trimethylolethane, hexane-1,2,6-triol, butane-1,2,4-triol, tris(β-hydroxyethyl)isocyanurate, pentaerythritol, mannitol or sorbitol.

Also suitable are polyols which contain further functional groups (oligomers or polymers). These are the hydroxyl-containing polyesters, polycarbonates, polycaprolactones, polyethers, polythioethers, polyesteramides, polyurethanes or polyacetals that are known per se. They possess a number-average molecular weight of 134 to 3500. The polyols are used alone or in mixtures.

The catalysts C) are organotin compounds of the following composition: RₙSnXₘ (II), in which R=alkyl radical having 1 to 10 carbon atoms and X=carboxylate radical of a carboxylic acid having 1 to 20 carbon atoms and n=1, 2 or 3, m=1, 2 or 3, and n+m=4. They are used in a concentration of from 0.01% to 3% by weight.

Particular suitability is possessed by catalysts such as butyltin tris(2-ethylhexanoate) and dibutyltin dilaurate.

The compounds D) are mono-, di-, and poly-functional carboxylic acids having 1 to 40 carbon atoms. Particularly suitable are acetic, propionic, n-octanoic, n-decanoic, n-dodecanoic, succinic, adipic acid, n-octanedioic and n-dodecanedioic acid, trimellitic, trimesic or pyromellitic acid. They are present in a concentration of from 0.1% to 5% by weight.

As polyisocyanates E), optionally, aromatic, aliphatic, (cyclo-)aliphatic and/or cycloaliphatic polyisocyanates having at least two NCO groups are reacted together with A) and B), particularly the following: isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate/2,4,4-trimethylhexamethylene diisocyanate (TMDI), norbornane diisocyanate (NBDI), toluidine diisocyanate (TDI), and/or methylenediphenyl diisocyanate (MDI) and also tetramethylxylylene diisocyanate (TMXDI) are used with preference. Very particular preference is given to IPDI, HDI, and H₁₂MDI. Additionally the polyisocyanates E) may contain further functional groups as well, such as isocyanurates, biurets or allophanates.

The compounds F), which may likewise be reacted together with A) and B), are monomeric monofunctional alcohols, monomeric monofunctional or difunctional amines and/or blocking agents. Suitable examples include methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols, and hydroxymethylcyclohexane. Additionally, dimethylamine, ethylamine, diethylamine, propylamine, dipropylamine, butylamine, dibutylamine, hexylamine, dihexylamine, ethylenediamine, propylenediamine, butylenediamine, hexamethylenediamine. Suitable blocking agents for NCO groups include all common compounds which can be eliminated again at temperatures below 200° C., such as methyl ethyl ketoxime, acetone oxime, phenol, ε-caprolactam, 1,2,4-triazole, 2,5-dimethylpyrazole, diethyl malonate, ethyl acetoacetate or diisopropylamine.

The reaction of the polyisocyanates A) carrying uretdione groups, and, if desired, polyisocyanates E) and/or F) to give the polyaddition compounds of the invention comprises the reaction of the free NCO groups of A) and, if desired, E) and/or F) with active-hydrogen-bearing compounds of B).

The invention also provides a process for solvent-free continuous preparation of low-viscosity polyaddition compounds containing uretdione groups and obtained by solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
   in which R=alkyl radical having 1 to 10 carbon atoms and X=carboxylate radical of a carboxylic acid having 1 to 20 carbon atoms and n=1, 2 or 3, m=1, 2 or 3 and n+m=4,
   in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, further aromatic, aliphatic, (cyclo-)aliphatic and/or cycloaliphatic polyisocyanates;
F) and optionally further monoalcohols, monoamines, diamines and/or blocking agents;
   wherein further auxiliaries and additives may be present,

in an extruder, flow tube, intensive compounder, intensive mixer or static mixer by intense commixing and short-duration reaction with heat supply at temperatures >50° C. and subsequent isolation of the end product by rapid cooling.

The principle of the process is that the reaction of the starting compounds takes place continuously, in particular in an extruder, flow tube, intensive compounder, intensive mixer or static mixer, by intense commixing and short-duration reaction with heat supply. This means that the residence time of the starting materials in the aforementioned equipment is usually 3 seconds to 15 minutes, preferably 3 seconds to 5 minutes, and more preferably 5 to 180 seconds. The reactants are reacted with short duration and with heat supply at temperatures of 50° C. to 325° C., preferably of 50 to 250° C., and very preferably of 70 to 220° C. Depending on the nature of the starting materials and of the end products, however, it is also possible for these residence time and temperature values to occupy other, preferred ranges. If desired, a continuous after reaction is included afterward. Subsequent rapid cooling then produces the end product.

Equipment particularly suitable for the process of the invention, and used with preference, includes extruders such as single-screw or multi-screw extruders, especially twin-screw extruders, planetary roller extruders or annular extruders, flow tubes, intensive compounders, intensive mixers, or static mixers.

The starting compounds are metered to the equipment generally in separate product streams. Where there are more than two product streams, these streams can also be supplied in bundle form. Different hydroxyl-containing starting materials can be combined into one product stream. It is also possible additionally to add catalysts and/or adjuvants such as flow control agents, or stabilizers, to this product stream. Similarly, polyisocyanates, and also the uretdione or uretdiones of polyisocyanates, can be combined with catalysts and/or adjuvants such as flow control agents or stabilizers into one product stream. The streams may also be divided and so supplied in different proportions to different sites in the equipment. In this way, in a targeted fashion, concentration gradients are set up, and this may induce the reaction to proceed to completion. The entry point of the product streams can be varied in sequence and offset in time.

For a preliminary reaction and/or for completion of the reaction it is also possible for two or more pieces of equipment to be combined.

The cooling downstream of the rapid reaction can be integrated in the reaction section, in the form of a multibarrel embodiment such as in the case of extruders or Conterna machines. The following may also be employed: tube bundles, tubular coils, chill rolls, air conveyors, metal conveyor belts, and water baths, with and without a downstream pelletizer.

The formulation is first of all brought to an appropriate temperature by means of further cooling using corresponding aforementioned apparatus, depending on the viscosity of the product leaving the intensive compounder zone or the after reaction zone. This cooling is followed by pelletizing or else by comminution to a desired particle size by means of a roll crusher, pin mill, hammer mill, flaking rolls, strand pelletizer (in combination with a water bath, for example), other pelletizers or similar.

The invention additionally provides for the use of the low-viscosity polyaddition compounds of the invention, containing uretdione groups, in thermoplastic polyurethanes (TPU) and molding compounds, polyurethane powder coating materials, and PU adhesives.

The invention further provides thermoplastic polyurethane molding compounds which contain low-viscosity polyaddition compounds containing uretdione groups and obtained by solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
   in which R=alkyl radical having 1 to 10 carbon atoms and X=carboxylate radical of a carboxylic acid having 1 to 20 carbon atoms and n=1, 2 or 3, m=1, 2 or 3 and n+m=4,
   in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, further aromatic, aliphatic, (cyclo-)aliphatic and/or cycloaliphatic polyisocyanates;
F) and optionally further monoalcohols, monoamines, diamines and/or blocking agents;
   and further polymers, auxiliaries and/or additives may be present.

For this purpose the polyaddition compounds of the invention containing uretdione groups can be blended with polymers, alternatively with polycarbonates, acrylonitrile copolymers, acrylonitrile-butadiene-styrene polymers, acrylonitrile-styrene-acrylic rubber molding compounds, copolymers of ethylene and/or propylene, and of acrylic acid or methacrylic acid or sodium salts or Zn salts thereof, copolymers of ethylene and/or propylene and also acrylic esters or methacrylic esters, and auxiliaries and additives such as, for example, UV stabilizers and antioxidants.

The molding compounds of the invention can be produced by mixing the TPU pellets, prepared by methods known in principle, with the respective adjuvants and compounding the mixture in a way which is known to the skilled worker, by reextrusion. Subsequently the resulting molding compound can be pelletized and converted by (cold) grinding to a sinterable powder suitable, for example, for processing by the powder slush process (see, for example, DE 39 32 923 or else U.S. Pat. No. 6,057,391). Such powders preferably have particle sizes of 50 to 500 μm. The molding compounds of the invention are suitable for producing a wide variety of moldings, examples including films and/or sintered sheets.

The films and/or sintered sheets produced from the polyurethane molding compounds of the invention are suitable for example for use as surface coverings in means of transport (e.g., aircraft, automobiles, ships, and railways).

The invention also provides polyurethane powder coating compositions substantially comprising
I. low-viscosity polyaddition compounds containing uretdione groups and obtained by solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
   in which R=alkyl radical having 1 to 10 carbon atoms and X=carboxylate radical of a carboxylic acid having 1 to 20 carbon atoms and n=1, 2 or 3, m=1, 2 or 3 and n+m=4,
   in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, further aromatic, aliphatic, (cyclo-)aliphatic and/or cycloaliphatic polyisocyanates;
F) and optionally further monoalcohols, monoamines, diamines and/or blocking agents;
   wherein further auxiliaries and additives may be present;

having a melting point of 40 to 130° C., a free NCO content of less than 5% by weight, and a uretdione content of 1% to 18% by weight;
II. optionally a hydroxyl-containing polymer having a melting point of 40 to 130° C. and an OH number of between 20 and 200 mg KOH/g;
III. optionally catalysts for accelerating the crosslinking reaction;
IV. optionally acid scavenger compounds;
   wherein further auxiliaries and additives may be present.

For the hydroxyl-containing polymers II. it is preferred to use polyesters, polyethers, polyacrylates, polyurethanes and/or polycarbonates having an OH number of 20 to 200 (in mg KOH/g). Particular preference is given to using polyesters having an OH number of 30 to 150, an average molecular weight of 500 to 6000 g/mol, and a melting point of between 40 and 130° C. Polyesters of this kind may be amorphous or (partially) crystalline. Such binders are described for example in EP 669 354 and EP 254 152. It will be appreciated that mixtures of such polymers can also be used.

Useful catalysts III. for accelerating the crosslinking reaction of the polymers are organometallic compounds such as, for example, dibutyltin dilaurate (DBTL) but also tertiary amines such as, for example, 1,4-diazabicyclo[2.2.2]octane (DABCO), 1,8-diazabicyclo[5.4.0]undec-7-ene (DBU), and 1,5-diazabicyclo[4.3.0]non-5-ene (DBN).

Further catalysts III. for accelerating the crosslinking reaction of the polyaddition compound containing uretdione groups with the hydroxyl-containing polymers are, in particular, metal acetylacetonates, metal hydroxides, metal alkoxides or quaternary ammonium salts with hydroxide, fluoride or carboxylate counterions. They are described for example in WO 00/34355, DE 103 20 267, DE 102 05 608, and DE 103 20 266.

The fraction of the catalyst or catalyst mixture as a proportion of the total amount of the powder coating formulation is 0.01% to 3% by mass.

The activity of the particularly efficient catalysts decreases significantly in the presence of acids. The conventional reaction partners of polyaddition compounds containing uretdione groups include hydroxyl-containing polyesters. Because of the way in which these polyesters are prepared, they occasionally still include acid groups to a small extent. The amount of acid groups in the polyesters should be below 20 mg KOH/g, since otherwise the catalysts are too greatly inhibited. In the presence of polyesters which carry such acid groups it is appropriate either to use the aforementioned catalysts in excess, relative to the acid groups, or else to add reactive compounds which are capable of scavenging acid groups. Both monofunctional and polyfunctional compounds can be used for this purpose.

Reactive acid scavenger compounds IV) are common knowledge in paint chemistry. For example, epoxy compounds, carbodiimides, hydroxyalkylamides or 2-oxazolines, but also inorganic salts such as hydroxides, hydrogen carbonates or carbonates, react with acid groups at elevated temperatures. Suitable examples include triglycidyl ether isocyanurate (TGIC), EPIKOTE 828 (diglycidyl ether based on bisphenol A, Shell), Versatic acid glycidyl esters, ethylhexyl glycidyl ether, butyl glycidyl ether, POLYPOX R 16 (pentaerythritol tetraglycidyl ether, UPPC AG), and also other Polypox grades containing free epoxy groups, VESTAGON EP HA 320, (hydroxyalkylamide, Degussa AG), but also phenylenebisoxazoline, 2-methyl-2-oxazoline, 2-hydroxyethyl-2-oxazoline, 2-hydroxypropyl-2-oxazoline, 5-hydroxypentyl-2-oxazoline, sodium carbonate, potassium carbonate, and calcium carbonate. It will be appreciated that mixtures of such substances are also suitable. These reactive compounds can be used in weight fractions of 0.1% to 10%, preferably of 0.5% to 3%, based on the total formulation.

For powder coating production it is possible to add the auxiliaries and additives that are customary in powder coating technology, such as flow control agents, polysilicones or acrylates for example, light stabilizers, sterically hindered amines for example, or other auxiliaries, as described for example in EP 0 669 353, in a total amount of 0.05% to 5% by weight. Fillers and pigments, such as titanium dioxide, for example, can be added in an amount of up to 50% by weight of the total composition.

Also suitable in addition are the catalysts which are customary for PU chemistry, examples being organometallic compounds such as DBTL, for example, but also tertiary amines such as 1,4-diazabicyclo[2.2.2]octane, DBU, and DBN, for example.

The invention further provides a process for producing polyurethane powder coating compositions in heatable equipment, with an upper temperature limit of 120 to 130° C.

All of the constituents for producing a powder coating composition can be homogenized in suitable equipment, such as heatable compounders, for example, but preferably by extrusion, in the course of which upper temperature limits of 120 to 130° C. ought not to be exceeded. After cooling to room temperature and appropriate comminution, the extruded mass is ground to form the ready-to-spray powder. Application of this powder to suitable substrates can take place by the known techniques, such as by electrostatic powder spraying or fluidized-bed sintering, with or without electrostatic assistance. Following powder application, the coated workpieces are cured by heating at a temperature of 120 to 220° C. for 4 to 60 minutes, preferably at 120 to 180° C. for 6 to 30 minutes.

The invention also provides polyurethane adhesive compositions substantially comprising
I. low-viscosity polyaddition compounds containing uretdione groups and obtained by solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
   in which R=alkyl radical having 1 to 10 carbon atoms and X=carboxylate radical of a carboxylic acid having 1 to 20 carbon atoms and n=1, 2 or 3, m=1, 2 or 3 and n+m=4,
   in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, further aromatic, aliphatic, (cyclo-)aliphatic and/or cycloaliphatic polyisocyanates;
F) and optionally further monoalcohols, monoamines, diamines and/or blocking agents;
   wherein further auxiliaries and additives may be present;

having a free NCO content of less than 5% by weight, and a uretdione content of 1% to 18% by weight;
II. optionally a hydroxyl-containing polymer having an OH number of between 20 and 200 mg KOH/g;
III. optionally catalysts for accelerating the crosslinking reaction;
IV. optionally acid scavenger compounds;
   wherein further auxiliaries and additives may be present.

For the hydroxyl-containing polymers II. it is preferred to use polyesters, polyethers, polyacrylates, polyurethanes and/or polycarbonates having an OH number of from 20 to 200 (in mg KOH/g). Particular preference is given to using polyesters having an OH number of from 30 to 150, an average molecular weight of 500 to 6000 g/mol. Polyesters of this kind may be amorphous or (partially) crystalline. Such binders are described for example in EP 0 669 354 and EP 0 254 152. It will be appreciated that mixtures of such polymers can also be used.

Useful catalysts III. for accelerating the crosslinking reaction of the polyaddition compound containing uretdione groups with the hydroxyl-containing polymers are organometallic compounds such as, for example, DBTL but also tertiary amines such as, for example, 1,4-diazabicyclo[2.2.2]octane, DBU, and DBN.

Useful catalysts III. for accelerating the crosslinking reaction of the polyaddition compound containing uretdione groups with the hydroxyl-containing polymers are, in particular, metal acetylacetonates, metal hydroxides, metal alkoxides or quaternary ammonium salts with hydroxide, fluoride or carboxylate counterions. They are described for example in WO 00/34355, DE 103 20 267, DE 102 05 608, and DE 103 20 266.

The fraction of the catalyst or catalyst mixture as a proportion of the total amount of the adhesive formulation is 0.01% to 3% by mass.

The activity of the particularly efficient catalysts decreases significantly in the presence of acids. The conventional reaction partners of polyaddition compounds containing uretdione groups include hydroxyl-containing polyesters. Because of the way in which these polyesters are prepared, they occasionally still include acid groups to a small extent. The amount of acid groups in the polyesters should be below 20 mg KOH/g, since otherwise the catalysts are too greatly inhibited. In the presence of polyesters which carry such acid groups it is appropriate either to use the aforementioned catalysts in excess, relative to the acid groups, or else to add reactive compounds which are capable of scavenging acid groups. Both monofunctional and polyfunctional compounds can be used for this purpose.

Reactive acid scavenger compounds IV) are common knowledge in chemistry. For example, epoxy compounds, carbodiimides, hydroxyalkylamides or 2-oxazolines, but also inorganic salts such as hydroxides, hydrogen carbonates or carbonates, react with acid groups at elevated temperatures. Suitable examples include triglycidyl ether isocyanurate (TGIC), EPIKOTE 828 (diglycidyl ether based on bisphenol A, Shell), Versatic acid glycidyl esters, ethylhexyl glycidyl ether, butyl glycidyl ether, POLYPOX R 16 (pentaerythritol tetraglycidyl ether, UPPC AG), and also other polypox grades containing free epoxy groups, VESTAGON EP HA 320, (hydroxyalkylamide, Degussa AG), but also phenylenebisoxazoline, 2-methyl-2-oxazoline, 2-hydroxyethyl-2-oxazoline, 2-hydroxypropyl-2-oxazoline, 5-hydroxypentyl-2-oxazoline, sodium carbonate, potassium carbonate, and calcium carbonate. It will be appreciated that mixtures of such substances are also suitable. These reactive compounds can be used in weight fractions of 0.1% to 10%, preferably of 0.5% to 3%, based on the total formulation.

For adhesive production it is possible to add the auxiliaries and additives that are customary in adhesive technology, such as flow control agents, polysilicones or acrylates for example, light stabilizers, sterically hindered amines for example, or other auxiliaries, as described for example in EP 0 669 353, in a total amount of 0.05% to 5% by weight. Fillers and pigments, such as titanium dioxide, for example, can be added in an amount of up to 50% by weight of the total composition.

Also suitable in addition are the catalysts which are customary for PU chemistry, examples being organometallic compounds such as DBTL, for example, but also tertiary amines such as 1,4-diazabicyclo[2.2.2]octane, DBU, and DBN, for example.

The subject matter of the invention is illustrated below with reference to examples.

### EXAMPLES

| Ingredients | Product description, manufacturer |
|---|---|
| IPDI uretdione (UD) | from IPDI by dimerization, free NCO content: |
| | 17.6%, latent NCO content: 20.0%; DEGUSSA AG |
| Diol | Hexanediol, Aldrich |
| Catalyst | Dibutyltin dilaurate, Aldrich |
| Carboxylic acid | Adipic acid, Aldrich |

Producing a Polyurethane Composition by the Process of the Invention

Three streams were employed:

Stream 1 was composed of hexanediol, or of a mixture of hexanediol and adipic acid.

Stream 2 was composed of the uretdione of isophorone diisocyanate (IPDI).

Stream 3 was composed of the catalyst, DBTL. The total amount, based on the total formula, was 0.10% or 0.15% respectively.

Stream 1 was fed as a melt at a rate of 2200 g/h into the first barrel of a twin-screw extruder (DSE 25) (stream temperature 70° C.).

Stream 2 was fed into the following barrel at a rate of 7630 g/h (stream temperature 80° C.).

Stream 3 was introduced through nozzles into stream 2 prior to entry into the extruder (10 or 15 g/h respectively).

The extruder used was composed of 8 barrels, which were separately heatable and coolable.

Barrel 1: 20-120° C., barrels 2-8: 90-160° C.

All temperatures represented setpoint temperatures. Regulation took place via electrical heating or water cooling. The die was likewise electrically heated. The screw speed was 250 rpm. The reaction product was cooled on a cooling belt and ground.

| | |
|---|---|
| Molar ratio OH:NCO | 7:6 |
| Throughput (kg/h) | 9.8 |
| Revolutions/minute | 250 |
| Exit temperature (° C.) | about 150 |

| | | Hexanediol/Adipic acid | Viscosity |
|---|---|---|---|
| Experiment | Catalyst | (% by weight) | (120° C.) [Pas] |
| 1 | DBTL (0.1%) | 97.5/2.5 | 6900 |
| 2 | DBTL (0.15%) | 96.2/3.8 | 8100 |
| 3* | DBTL (0.1%) | 100/0 | 15000 |
| 4* | DBTL (0.15%) | 100/0 | 16000 |

*noninventive comparative examples

The polyaddition compounds of the invention are significantly (<40%) lower in their melt viscosity than the comparative examples catalyzed with DBTL.

1. A low-viscosity polyaddition compound containing uretdione groups and obtained by a solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ (II)
in which R is an alkyl radical having 1 to 10 carbon atoms, X is a carboxylate radical of a carboxylic acid having 1 to 20 carbon atoms, n is 1, 2 or 3, m is 1, 2 or 3 and n+m=4,
in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, additional aromatic, aliphatic, (cyclo-)aliphatic and/or cycloaliphatic polyisocyanates;
F) and optionally additional monoalcohols, monoamines, diamines and/or blocking agents;
wherein additional auxiliaries and additives may be present.

2. A low-viscosity polyaddition compound containing uretdione groups as claimed in claim 1,
wherein component A) is selected from the group consisting of
isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate/2,4,4-trimethylhexamethylene diisocyanate (TMDI), norbornane diisocyanate (NBDI), toluidine diisocyanate (TDI), methylenediphenyl diisocyanate (MDI), and/or tetramethylxylylene diisocyanate (TMXDI).

3. A low-viscosity polyaddition compound containing uretdione groups as claimed in claim 2,
wherein component A) is selected from the group consisting of
IPDI, HDI and/or H₁₂MDI.

4. A low-viscosity polyaddition compound containing uretdione groups as claimed in claim 1,
wherein component B) is selected from the group consisting of
ethylene glycol, triethylene glycol, butane-1,4-diol, pentane-1,5-diol, hexane-1,6-diol, 3-methylpentane-1,5-diol, neopentyl glycol, 2,2,4(2,4,4)-trimethylhexanediol, neopentyl glycol hydroxypivalate, trimethylolpropane, ditrimethylolpropane, trimethylolethane, hexane-1,2,6-triol, butane-1,2,4-triol, tris(β-hydroxyethyl)isocyanurate, pentaerythritol, mannitol, sorbitol, hydroxyl-containing polyesters, polycarbonates, polycaprolactones, polyethers, polythioethers, polyesteramides, polyurethanes and/or polyacetals, alone or in a mixture.

5. A low-viscosity polyaddition compound containing uretdione groups as claimed in claim 1,
wherein component C) is selected from the group consisting of
butyltin tris(2-ethylhexanoate) and/or dibutyltin dilaurate.

6. A low-viscosity polyaddition compound containing uretdione groups as claimed in claim 1,
wherein component D) is selected from the group consisting of
acetic, propionic, n-octanoic, n-decanoic, n-dodecanoic, succinic, adipic acid, n-octanedioic and n-dodecanedioic acid, trimellitic, trimesic and/or pyromellitic acid.

7. A low-viscosity polyaddition compound containing uretdione groups as claimed in claim 1,
wherein component E) is selected from the group consisting of
isophorone diisocyanate (IPDI), hexamethylene diisocyanate (HDI), diisocyanatodicyclohexylmethane (H₁₂MDI), 2-methylpentane diisocyanate (MPDI), 2,2,4-trimethylhexamethylene diisocyanate/2,4,4-trimethylhexamethylene diisocyanate (TMDI), norbornane diisocyanate (NBDI), toluidine diisocyanate (TDI), methylenediphenyl diisocyanate (MDI) and/or tetramethylxylylene diisocyanate (TMXDI), alone or in a mixture, are used as component E).

8. A low-viscosity polyaddition compound containing uretdione groups as claimed in claim 7,
wherein component E) is selected from the group consisting of
isocyanurates, biurets and/or allophanates are used.

9. A low-viscosity polyaddition compound containing uretdione groups as claimed in claim 1,
wherein component F) is selected from the group consisting of
methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols, hydroxymethylcyclohexane, dimethylamine, ethylamine, diethylamine, propylamine, dipropylamine, butylamine, dibutylamine, hexylamine, dihexylamine, ethylenediamine, propylenediamine, butylenediamine, hexamethylenediamine, methyl ethyl ketoxime, acetone oxime, phenol, ε-caprolactam, 1,2,4-triazole, 2,5-dimethylpyrazole, diethyl malonate, ethyl acetoacetate, diisopropylamine, alone or in a mixture, are used as compounds F).

10. A process for solvent-free continuous preparation of a low-viscosity polyaddition compound containing uretdione groups and obtained by a solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
in which R is an alkyl radical having 1 to 10 carbon atoms, X is a carboxylate radical of a carboxylic acid having 1 to 20 carbon atoms, n is 1, 2 or 3, m is 1, 2 or 3 and n+m=4,
in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, additional aromatic, aliphatic, (cyclo-)aliphatic and/or cycloaliphatic polyisocyanates;
F) and optionally additional monoalcohols, monoamines, diamines and/or blocking agents;
wherein additional auxiliaries and additives may be present,
in an extruder, flow tube, intensive compounder, intensive mixer or static mixer by intense commixing and short-duration reaction with heat supply at temperatures >50° C. and subsequent isolation of the end product by rapid cooling.

11. A process as claimed in claim 10,
wherein
the residence time of the starting materials is 3 seconds to 15 minutes.

12. A process as claimed in claim 10,
wherein
the reaction takes place in a single-screw, twin-screw or multi-screw extruder, annular extruder or planetary roller extruder.

13. A process as claimed in claim 12,
wherein
the reaction takes place in a twin-screw extruder.

14. A process as claimed in claim 10,
wherein
the reaction takes place in a flow tube, intensive mixer or intensive compounder.

15. A process as claimed in claim 10,
wherein
the reaction takes place in a static mixer.

16. A process as claimed in claim 11,
wherein
the reaction takes place in an extruder, intensive compounder, intensive mixer or static mixer having two or more identical or different barrels which can be thermally controlled independently of one another.

17. A process as claimed in claim 10,
wherein
the temperature in the extruder, intensive compounder, intensive mixer or static mixer is 50 to 325° C.

18. A process as claimed in claim 10,
wherein
by appropriate equipping of the mixing chambers and configuration of the screw geometry the extruder or intensive compounder on the one hand leads to an intense and rapid commixing and rapid reaction in conjunction with intense heat exchange and on the other hand brings about uniform flow in the longitudinal direction with an extremely uniform residence time.

19. A process as claimed in claim 10,
wherein
the starting materials and/or catalysts and/or adjuvants are supplied together or in separate product streams, in liquid or solid form, to the extruder, flow tube, intensive compounder, intensive mixer or static mixer.

20. A process as claimed in claim 19,
wherein
the adjuvants are combined with the starting materials into one product stream.

21. A thermoplastic polyurethane (TPU) or molding compound, polyurethane powder coating material or PU adhesive comprising a low-viscosity polyaddition compound containing uretdione groups as claimed in claim 1.

22. A thermoplastic polyurethane molding compound which contains
I. a low-viscosity polyaddition compound containing uretdione groups and obtained by a solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙSnXₘ
in which R is an alkyl radical having 1 to 10 carbon atoms, and X is a carboxylate radical of a carboxylic acid having 1 to 20 carbon atoms, n is 1, 2 or 3, m is 1, 2 or 3 and n+m=4,
in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, additional aromatic, aliphatic, (cyclo-)aliphatic and/or cycloaliphatic polyisocyanates;
F) and optionally additional monoalcohols, monoamines, diamines and/or blocking agents;
wherein additional polymers, auxiliaries and additives may be present;
having a melting point of 40 to 130° C., a free NCO content of less than 5% by weight, and a uretdione content of 1% to 18% by weight;
II. optionally a hydroxyl-containing polymer having a melting point of 40 to 130° C. and an OH number of between 20 and 200 mg KOH/g;
III. optionally catalysts for accelerating the crosslinking reaction;
IV. optionally acid scavenger compounds; and
wherein additional auxiliaries and additives may be present.

23. A polyurethane powder coating composition substantially comprising
I. a low-viscosity polyaddition compound containing uretdione groups and obtained by a solvent-free reaction at temperatures above 50° C. of
A) at least one aromatic, aliphatic, (cyclo-)aliphatic and/or cycloaliphatic polyisocyanate containing uretdione groups and having at least two NCO groups and
B) at least one monomeric, oligomeric and/or polymeric polyol having at least two OH groups;
C) in the presence of organotin compounds of composition RₙS Xₘ
in which R is an alkyl radical having 1 to 10 carbon atoms, X is a carboxylate radical of a carboxylic acid having 1 to 20 carbon atoms, n is 1, 2 or 3, m is 1, 2 or 3 and n+m=4,
in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, additional aromatic, aliphatic, (cyclo-)aliphatic and/or cycloaliphatic polyisocyanates;
F) and optionally additional monoalcohols, monoamines, diamines and/or blocking agents;
wherein additional auxiliaries and additives may be present;
having a melting point of 40 to 130° C., a free NCO content of less than 5% by weight, and a uretdione content of 1% to 18% by weight;
II. optionally a hydroxyl-containing polymer having a melting point of 40 to 130° C. and an OH number of between 20 and 200 mg KOH/g;
III. optionally catalysts for accelerating the crosslinking reaction;
IV. optionally acid scavenger compounds; and
wherein additional auxiliaries and additives may be present.

24. A polyurethane adhesive composition substantially comprising
I. a low-viscosity polyaddition compound containing uretdione groups and obtained by a solvent-free reaction at temperatures above 50° C. of A) at least one aromatic, aliphatic, (cyclo-)aliphatic and/or cycloaliphatic
polyisocyanate containing uretdione groups and having at least two NCO groups and
B) at least one monomeric, oligomeric and/or polymeric polyol having at least two OH groups;
C) in the presence of organotin compounds of composition RₙSNXₘ
in which R is an alkyl radical having 1 to 10 carbon atoms, X is a carboxylate radical of a carboxylic acid having 1 to 20 carbon atoms, n is 1, 2 or 3, m is 1, 2 or 3 and n+m=4,
in a concentration of from 0.01 to 3% by weight, based on the total composition, and
D) in the presence of monocarboxylic, dicarboxylic or polycarboxylic acids in a concentration of 0.1%-5% by weight, based on polyol B);
E) and/or, optionally, ef additional aromatic, aliphatic, (cyclo-)aliphatic and/or cycloaliphatic polyisocyanates;
F) and optionally additional monoalcohols, monoamines, diamines and/or blocking agents;
wherein additional auxiliaries and additives may be present;
having a free NCO content of less than 5% by weight, and a uretdione content of 1% to 18% by weight;
II. optionally a hydroxyl-containing polymer having an OH number of between 20 and 200 mg KOH/g;
III. optionally catalysts for accelerating the crosslinking reaction; and
IV. optionally acid scavenger compounds;
wherein additional auxiliaries and additives may be present.

25. A composition as claimed in claim 22,
wherein component II is selected from the group consisting of
polyesters, polyethers, polyacrylates, polyurethanes and/or polycarbonates having an OH number of 20 to 200 (in mg KOH/g) are used as cempenent II.

26. A composition as claimed in claim 22,
wherein component III is selected from the group consisting of
DBTL but also tertiary amines such as 1,4-diazabicyclo[2.2.2]octane, DBU, and DBN, for example, metal acetylacetonates, metal hydroxides, metal alkoxides or quaternary ammonium salts with hydroxide, fluoride or carboxylate counterions.

27. A composition as claimed in claim 22,
wherein component IV is selected from the group consisting of
epoxy compounds, carbodiimides, hydroxyalkylamides or 2-oxazolines, organic salts such as hydroxides, hydrogen carbonates or carbonates with acid groups.

28. A composition as claimed in claim 23,
wherein component II is selected from the group consisting of
polyesters, polyethers, polyacrylates, polyurethanes and/or polycarbonates having an OH number of 20 to 200 (in mg KOH/g).

29. A composition as claimed in claim 23,
wherein component III is selected from the group consisting of
DBTL but also tertiary amines such as 1,4-diazabicyclo[2.2.2]octane, DBU, and DBN, for example, metal acetylacetonates, metal hydroxides, metal alkoxides or quaternary ammonium salts with hydroxide, fluoride or carboxylate counterions.

30. A composition as claimed in claim 23,
wherein component IV is selected from the group consisting of
epoxy compounds, carbodiimides, hydroxyalkylamides or 2-oxazolines, organic salts such as hydroxides, hydrogen carbonates or carbonates with acid groups.

31. A composition as claimed in claim 24,
wherein component II is selected from the group consisting of
polyesters, polyethers, polyacrylates, polyurethanes and/or polycarbonates having an OH number of 20 to 200 (in mg KOH/g).

32. A composition as claimed in claim 24,
wherein component III is selected from the group consisting of
DBTL but also tertiary amines such as 1,4-diazabicyclo[2.2.2]octane, DBU, and DBN, for example, metal acetylacetonates, metal hydroxides, metal alkoxides or quaternary ammonium salts with hydroxide, fluoride or carboxylate counterions.

33. A composition as claimed in claim 24,
wherein component IV is selected from the group consisting of
epoxy compounds, carbodiimides, hydroxyalkylamides or 2-oxazolines, organic salts such as hydroxides, hydrogen carbonates or carbonates with acid groups.
